(19) United States
(12) Patent Application Publication
(10) Pub. No.: US 2004/0204743 A1
(43) Pub. Date: Oct. 14, 2004

(54) Remotely operating external medical devices

(76) Inventors: Thomas J. McGrath, Everett, WA (US); Randy L. Merry, Woodinville, WA (US); Mark P. Moore, Redmond, WA (US)

Correspondence Address: SHUMAKER & SIEFFERT, P. A., 8425 SEASONS PARKWAY, SUITE 105, ST. PAUL, MN, 55125, US

(21) Appl. No.: 10/756,895

(22) Filed: Jan. 14, 2004

Related U.S. Application Data
(60) Provisional application No. 60/440,320, filed on Jan. 14, 2003.

(51) Int. Cl. A61N 1/18

(52) U.S. Cl. 607/5; 340/825.72; 700/17

(57) ABSTRACT

In general, the invention provides techniques for remotely controlling an external medical device with a remote device via a wireless communication session. The remote device sends one or more commands to the external medical device via the wireless communication session, and the external medical device carries out the commands. The commands may pertain to various functions such as changing a display, applying a therapy, configuring the therapy, initiating data transmission to a remote location and updating medical event information.

This application claims the benefit of U.S. Provisional Application Serial No. 60/440,320, filed Jan. 14, 2003, the entire content of which is incorporated herein by reference.

### TECHNICAL FIELD

The invention relates to medical devices, and more particularly, to medical device communication.

### BACKGROUND

An external defibrillator is a device that stores energy, typically in one or more high-voltage capacitors, and delivers the stored energy to a patient. The defibrillator delivers energy to a heart that is undergoing ventricular fibrillation and has lost its ability to contract. Ventricular fibrillation is particularly life-threatening because activity within the ventricles of the heart is so uncoordinated that virtually no pumping of blood takes place. If untreated, the patient whose heart is undergoing fibrillation may die within a matter of minutes.

An electrical pulse delivered to a fibrillating heart may depolarize the heart and cause the heart to reestablish a normal sinus rhythm. An external defibrillator applies a defibrillation pulse via electrodes placed upon the chest of the patient. When a switch is closed, the defibrillator delivers at least some of the stored energy to the patient. In some cases, the patient may need multiple shocks, and different quantities of energy may be delivered with each shock.

Some defibrillators also monitor the patient via the electrodes, and generate a record of the condition and treatment of the patient. For example, the defibrillator may record an electrocardiogram (ECG) of the patient sensed via the defibrillation electrodes or sensed via a specialized set of ECG electrodes. In addition, some defibrillators keep track of the therapy provided to the patient by recording data about the defibrillation pulses delivered to the patient and the time at which these pulses were delivered. The defibrillator may also include a microphone to make an audio recording of the treatment of the patient. Information surrounding the treatment of the patient, i.e., medical event information, may be stored within a memory of the defibrillator.

At the scene of a medical emergency, emergency personnel may operate a plurality of external medical devices. Emergency personnel, such as police officers, firefighters and emergency medical technicians, may employ medical devices such as automated external defibrillators (AEDs), full-featured external defibrillators, drug delivery devices, and personal monitors. Each device may record respective medical event information pertaining to the monitoring or treatment of the patient.

### SUMMARY

In general, the invention provides techniques for remotely operating an external medical device, such as a defibrillator, with a remote device, such as a tablet computer or personal digital assistant. The external medical device and the remote device establish a wireless communication session. The remote device sends one or more commands to the external medical device via the wireless communication session, and the external medical device carries out the commands. The commands may pertain to administration of therapy, for example, collection of medical event information, or changes to the settings of the external medical device.

In some embodiments of the invention, the computing device may be configured to generate a medical record or “run report” that includes information about the patient, the condition of the patient or treatment of the patient. The external medical device and the remote device may exchange medical event information via the wireless communication session that may be used in the run report. The remote device may generate a run report based on medical event information received from the external medical device.

In one embodiment, the invention is directed to a method comprising establishing a wireless communication session with an external medical device and sending a command to the external medical device via the wireless communication session to remotely operate the external medical device. A remote device carrying out this method may, for example, send a command to cause the external medical device to apply a therapy.

In another embodiment, the invention is directed to a method comprising establishing a wireless communication session between an external medical device and a remote device, receiving a command from the remote device via the wireless communication session and carrying out the command to operate the external medical device.

The invention also encompasses embodiments directed to a computer-readable medium containing instructions that cause a machine to carry out any of the methods of the invention.

In a further embodiment, the invention is directed to a device comprising a transceiver to establish a wireless communication session with an external medical device and a processor to control the transceiver to send a command to the medical device via the wireless communication session to remotely operate the external medical device. The device may also be configured to receive medical event information via the wireless communication session, and the processor may be configured to generate a run report based on the received medical event information.

In an additional embodiment, the invention is directed to a device comprising a transceiver to establish a wireless communication session with a remote device and to receive a command from the remote device, and a processor to carry out the command. The device may include, for example, an electrocardiograph, a capnograph, a plethysmograph, a heart rate monitor, a temperature monitor, a blood oxygen monitor, a blood pressure monitor, an external defibrillator, an automated external defibrillator (AED), a drug delivery device, and an automated chest thumper.

In another embodiment, the invention is directed to a system comprising an external medical device and a remote device. The external medical device is configured to carry out commands sent via a wireless communication session with the remote computing device.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a block diagram illustrating an example environment in which a remote device wirelessly operates an external defibrillator.

FIG. 2 is a block diagram illustrating an example external defibrillator of FIG. 1 in more detail.

FIG. 3 is a block diagram illustrating an example computer of FIG. 1 in more detail.

FIG. 4 is a flow chart illustrating an exemplary process by which an external medical device executes commands issued by a remote device via a wireless communication session.

FIG. 5 is a flow chart illustrating an exemplary process by which a remote device remotely controls an external medical device via a wireless communication session.

### DETAILED DESCRIPTION

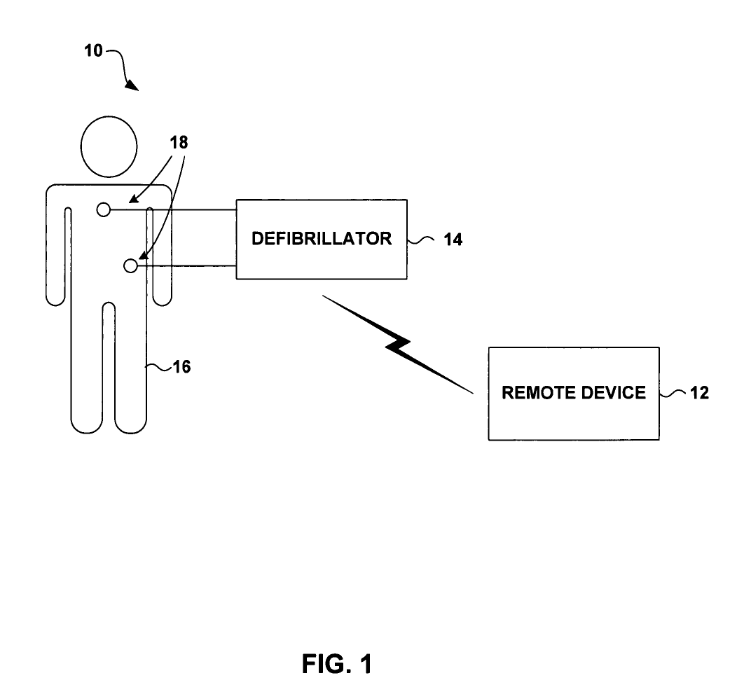

FIG. 1 is a block diagram illustrating an example environment 10 in which a remote device 12 wirelessly operates an external medical device 14. In the example of FIG. 1, external medical device 14 is a defibrillator. The invention is not limited to application with a single external medical device, however, nor is the invention limited to application with a defibrillator. Rather, the invention may be applicable to combinations of two or more external medical devices or combinations of one or more external medical devices and one or more remote devices. External medical devices include, but are not limited to, defibrillators such as defibrillator 14, drug delivery devices, automated external defibrillators (AEDs), automated chest thumpers, and the like.

In the example of FIG. 1, remote device 12 is a computer such as a tablet computer. The invention is not limited to applications in which remote device 12 includes computing capability. Remote devices include, but are not limited to, computers, personal digital assistants (PDAs), cellular telephones and the like. Remote device 12 may also be a dedicated remote device, or another external medical device.

As illustrated in FIG. 1, remote device 12 is capable of wireless communication and wirelessly communicates with defibrillator 14. In some embodiments, when both remote device 12 and defibrillator 14 are turned on and proximate to one another such that wireless communication is possible, one of remote device 12 or defibrillator 14 detects the other via a wireless communication medium and initiates wireless communication. Remote device 12 and defibrillator 14 establish a wireless communication session via the wireless communication medium in order to communicate with each other. In other embodiments, remote device 12 and defibrillator 14 do not detect one another before establishing a wireless communication session. When remote device 12 and defibrillator 14 are configured to communicate via a cellular telephone network, for example, communication may be established without proximity or detection.

A typical wireless communication medium is a radio frequency (RF) communication medium, and remote device 12 and defibrillator 14 can establish the local wireless communication session according to any of a number of local wireless communication standards. For example, remote device 12 and defibrillator 14 may establish a Bluetooth session according to the Bluetooth specification set, which was promulgated by the Bluetooth Special Interest Group (SIG), and is available for download at http://www.bluetooth.org. As another example, remote device 12 and defibrillator 14 may establish a wireless local area networking session, such as an IEEE 802.11A session, an IEEE 802.11B session, or an IEEE 802.11 G session according to the 802.11 specifications set promulgated by the Institute of Electrical and Electronics Engineers (IEEE).

Remote device 12 and defibrillator 14, in another example, may employ wireless communication based upon infrared light. Remote device 12 or defibrillator 14 can establish wireless communication session via infrared ports (not shown) that transmit and/or receive infrared light waves. Remote device 12 or defibrillator 14 may establish a wireless communication session via infrared light in accordance with specification sets promulgated by the Infrared Data Association (IrDA).

Pre-registration typically is not necessary in order to establish a local wireless communication session according to the Bluetooth, 802.11 or IrDA specification sets. In other words, remote device 12 and defibrillator 14 may be previously unknown to each other, and may establish an ad hoc network according to one of these specification sets. The ability to establish an ad hoc network allows computing devices, such as remote device 12 to control external medical devices, such as defibrillator 14, that are unknown to each other.

In yet another embodiment, remote device 12 and defibrillator 14 establish a wireless communication session via an intermediate network. Remote device 12 and defibrillator 14 may establish a wireless communication session via a cellular telephone network, for example. Remote device 12 or defibrillator 14 may establish the session by one “calling” the other. In this embodiment, remote device 12 and defibrillator 14 do not need to be proximate to one another to establish the wireless communication session.

Upon establishing a wireless communication session via one of the wireless communication mediums discussed above, remote device 12 sends one or more commands to defibrillator 14. Generally, an operator of remote device 12 interacts with remote device 12 via a user interface (not shown) to wirelessly send one or more commands to defibrillator 14. Defibrillator 14 carries out or executes the commands received from remote device 12.

The user interface of remote device 12, in some embodiments, may correspond to the user interface of the external medical device receiving the commands. Remote device 12 may, for example, include a push-button interface that is similar to the user interface of defibrillator 14, or remote device 12 may display a touchscreen interface that includes a graphical representation of the commands of the user interface of defibrillator 14.

Remote device 12 receives one or more inputs from an operator in response to the operator interacting with the user interface (not shown) of remote device 12. The user interface includes input-output devices, such as a mouse, a keyboard, a touchscreen, a CRT, a LED display, an LCD display, a microphone or a speaker. In response to receiving inputs from the operator, remote device 12 sends one or more commands to defibrillator 14. For example, the operator may select a “deliver therapy” option displayed on a screen of the user interface. Remote device 12 receives the selection and sends a command to defibrillator 14 to apply a therapy, such as a defibrillation pulse. Defibrillator 14 carries out the command.

A command sent by remote device 12 may instruct defibrillator 14 to perform one or more actions, such as changing a display presented to an operator, delivering a therapy to patient 16, initiating collection of vital signs associated with patient 16, or initiating data transmission to a remote location such as a hospital. A command may also instruct defibrillator 14 to configure a therapy by, for example, setting an amplitude, a width, a shape, and a period of the defibrillation pulse. A command may also direct defibrillator 14 to updating medical event information stored with defibrillator 14, as described below. A command from remote device may also direct defibrillator 14 to perform other actions associated with monitoring or treating patient 16.

Defibrillator 14, upon receiving the command, carries out the command. In some embodiments, defibrillator 14 wirelessly communicates via the established wireless communication session an acknowledgement of a command. The acknowledgement may include a report that the command had been carried out.

While executing the commands and treating patient 16, defibrillator 14, in some embodiments, stores medical event information in a memory (not shown) included in defibrillator 14. “Medical event information” includes any information pertaining to the patient, the condition of the patient or treatment of the patient. Defibrillator 14, in these embodiments, wirelessly communicates the medical event information to computer 12. Remote device 12 can use the medical event information to construct a “run report.” As used herein, a “run report” is any record that includes information about the patient, the condition of the patient or treatment of the patient.

For example, remote device 12 may receive medical event information from defibrillator 14 pertaining to delivery of a defibrillation pulse. The medical event information may include, for example, a time the pulse was applied, an amplitude or energy level, a width, a shape, and the response of the patient to the therapy. Using this medical event information, remote device 12 constructs a run report that includes information about delivery of the defibrillation pulse. Defibrillator 14 may collect other medical event information via electrodes 18, such as medical event information about the heart rate and rhythm of the patient. Remote device 12 may construct a run report that includes this medical event information as well.

Other external medical devices may record other medical event information and may send the medical information to remote device 12. A blood pressure monitor may send information about blood pressure, a temperature monitor may send information about temperature, a full-featured defibrillator may send an electrocardiogram, a capnograph may send information about the patient's respiration, and so on.

Remote device 12 can present the run report or portion thereof on the user interface so that the operator can analyze or edit the run report. In some embodiments, an operator may enter some medical event information into remote device 12, such as the patient's name, the patient's address, the patient's telephone number, the patient's social security number, the patient's ethnicity, the patient's age, the patient's weight, the location where the patient was found, the time of arrival, the patient's insurance coverage, the patient's chief complaint, the patient's current medications, the patient's allergies, and so on. Emergency medical technicians often include medical event information in the run reports that document their prehospital care.

In some embodiments, remote device 12 wirelessly communicates medical event information entered by an operator into defibrillator 14. Defibrillator 14 utilizes the medical event information to, for example, determine an appropriate therapy to apply. If the medical event information received from remote device 12 shows that the patient is a pediatric patient, for example, defibrillator 14 may employ energy delivery suitable for a pediatric patient. Remote device 12 and defibrillator 14 can exchange information, such as medical event information, without receiving a specific command from an operator to communicate the information.

Because medical event information can be wirelessly communicated from remote device 12 to defibrillator 14 and vice versa, an operator does not have to enter the same medical event information more than once. In some circumstances, the operator does not have to enter medical event information at all, because remote device 12 or defibrillator 14 records the information automatically. As a result, the operator experiences increased efficiency while preparing run reports.

Wireless communication also allows for remote device 12 to remotely and wirelessly control defibrillator 14. In some circumstance, remote control of defibrillator 14 may provide a measure of safety, since an operator of defibrillator 14 can maintain isolation from defibrillator 14. Isolation may also be a benefit when operating other external medical devices as well.

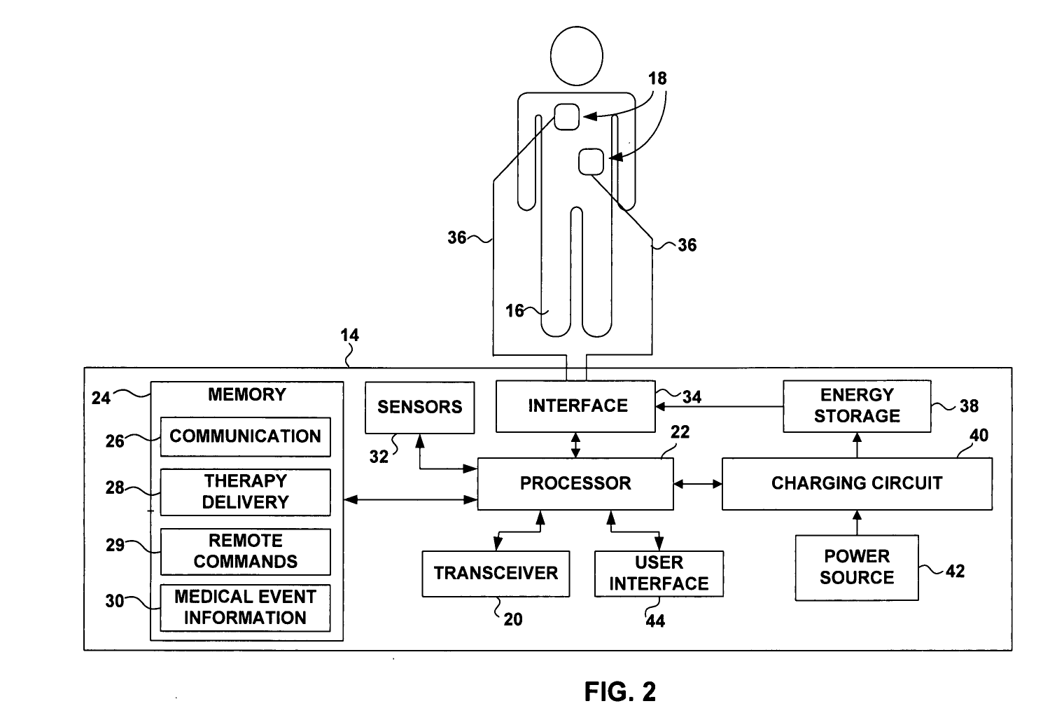

FIG. 2 is a block diagram illustrating an example external defibrillator 14 of FIG. 1 in more detail. Defibrillator 14 is subject to wireless control by a remote device. As illustrated in FIG. 2, defibrillator 14 includes a transceiver 20 for wireless communication. In one embodiment, transceiver 20 includes an antenna (not shown) to facilitate wireless communication via a radio frequency communication medium. In this embodiment, transceiver 20 takes, for example, the form of an integrated circuit or PCIMCA card with circuitry configured for wireless communication, such as a wireless network interface card. The wireless network interface card can be, in this embodiment, compliant with one or more wireless communication standards, such as the Bluetooth or 802.11 specification sets. In another embodiment, transceiver 20 takes the form of IrDA port for communication via the wireless communication medium that includes infrared light and compliant with IrDA specification sets. In another embodiment, transceiver 20 contains circuitry similar to a cellular telephone to communicate via a RF wireless communication medium.

A processor 22 coupled to transceiver 20 controls operation of transceiver 20. Processor 22 controls transceiver 20 and causes transceiver 20 to detect the presence of a remote device, establish a wireless communication session, receive commands sent by a remote device, transmit acknowledgments in response to the received commands, transmit medical event information and receive medical event information. Processor 22 also controls the operation of defibrillator 14 in monitoring patient 16, providing therapy to patient 16 and generating medical event information during the treatment of patient 16. In some embodiments, processor 22 takes the form of a microprocessor or an application specific integrated circuit (ASIC).

Processor 22 accesses memory 24 and executes instructions stored within memory 24. Memory 24 may include any of a variety of solid state, magnetic or optical media, such as RAM, ROM, CD-ROM, magnetic disk, or EEPROM. Memory 24 stores communication program instructions 26, therapy delivery program instructions 28, and remote commands 29. Communication program instructions 26 cause processor 22 to establish a wireless communication session and wirelessly communicate with other external medical or remote computing devices. Communication program instructions 26 may further cause processor 22 to detect the presence of a proximate remote device. Therapy delivery program instructions 28 cause processor 22 to monitor patient 16, evaluate the hear rhythm of patient 16 and, when appropriate, deliver defibrillation pulses to patient 16. Remote commands 29 comprise one or more commands received from computer 12 via an established wireless communication session. Remote commands 29 cause processor 22 to perform one or more of the actions discussed above, such as delivering a therapy.

Memory 24 also stores medical event information 30. Medical event information 30, in some instances, includes an ECG of patient 16 generated based on the electrical activity sensed via electrodes 18. Medical event information 30 can also include information about therapy delivered to patient 18. Medical event information 30 can further include an audio recording recorded during the treatment of patient 16 or any patient information received from any source. Medical event information 30 may include, for example, a capnograph or carbon dioxide measurements, a plethysmograph, a heart rate, a pulse rate, a blood oxygen saturation measurement, a blood pressure, a respiration rate, and the like. This information may be received from another medical device. This information may also be received via one or more sensors 32.

Sensors 32 can include, for example, one or more of a temperature sensor, a microphone, an ECG monitor, such as a Holter monitor, an oxygen sensor, a carbon dioxide sensor, a respiratory sensor, and a blood pressure monitor. Sensor 32 can also include an expanded electrode set, such as a twelve-, five- or three-lead electrode set. An expanded electrode set can be used to detect ECG signals more efficiently than electrodes 18, and can be used to provide a more thorough analysis of a condition of the heart of patient 16. Sensors 32 transmit sensed data to processor 22, which in turn analyzes the data and stores the data to memory 24 as medical event information 30.

Although defibrillator 14 is shown coupled to patient 16, remote device 12 does not require defibrillator 14 to be coupled to patient 16 in order for remote device 12 to wirelessly communicate with defibrillator 14.

Electrodes 18, in some embodiments, include hand-held electrode paddles or adhesive electrode pads placed on the skin of patient 16. Electrodes 18 are coupled to defibrillator 14 via conductors 36 and interface 34. Typically, interface 36 includes a receptacle and conductors 32 plug into the receptacle.

Interface 34 includes a switch (not shown in FIG. 2) that, when activated, couples an energy storage circuit 38 to electrodes 18. Energy storage circuit 38 stores the energy to be delivered to patient 16 in the form of a defibrillation pulse. The switch typically conforms to a conventional design and is formed, for example, of electrically operated relays. Alternatively, the switch, in some embodiments, comprises an arrangement of solid-state devices such as silicon-controlled rectifiers or insulated gate bipolar transistors.

Energy storage circuit 38 includes components, such as one or more capacitors, that store the energy to be delivered to patient 16 via electrodes 18. Before a defibrillation pulse can be delivered to patient 16, energy storage circuit 38 must be charged. Processor 22 directs a charging circuit 40 to charge energy storage circuit 38 to a high voltage level. Charging circuit 40 comprises, for example, a flyback charger that transfers energy from a power source 42 to energy storage circuit 38.

As in most conventional defibrillators, defibrillator 14 comprises a user interface 44 for directing operation of defibrillator 14. An operator of defibrillator 14 may interact with defibrillator 14 via user interface 44, which may include input-output devices such as a keypad, and buttons, dials, touchscreen, indicator lights, a CRT, LED display, LCD screen, or a speaker.

The operator may also interact with defibrillator 14 via remote device 12. The operator may use remote device 12 to send one or more commands to defibrillator 14 via a wireless communication session. Defibrillator 14 receives the commands and stores the received commands in memory 24 as remote commands 29. Processor 22 generally executes or carries out the commands. For example, an operator may use remote device 12 to send a command to defibrillator 14, directing defibrillator 14 to begin charging. In response, processor 22 controls charging circuit 40 to begin charging energy storage circuit 38. As another example the operator may use remote device 12 to send commands to defibrillator 14 to configure a therapy, such as commands to set an energy level for a patient based upon the patient's size.

When the energy stored in energy storage circuit 38 reaches the desired energy level, processor 22 may control transceiver 20 to provide an indication to the operator of remote device 12 that defibrillator 14 is ready to deliver therapy to patient 16. The operator may use remote device 12 to direct defibrillator 14 to deliver the therapy. Upon receiving a command to deliver therapy, processor 22 activates the switch to electrically connect energy storage circuit 36 to electrodes 18, and thereby deliver the defibrillation pulse to patient 16.

In some embodiments, processor 22 stores in memory 24 a record of the executed commands as medical event information 30. Processor 22 may further store in memory 24 a record of the response of patient 16 to therapy. Processor 22, in some embodiments, communicates the medical event information 30 to remote device 12 via the established wireless communication session. Processor 22 also may send medical event information 30 to any number of other external medical devices or remote devices.

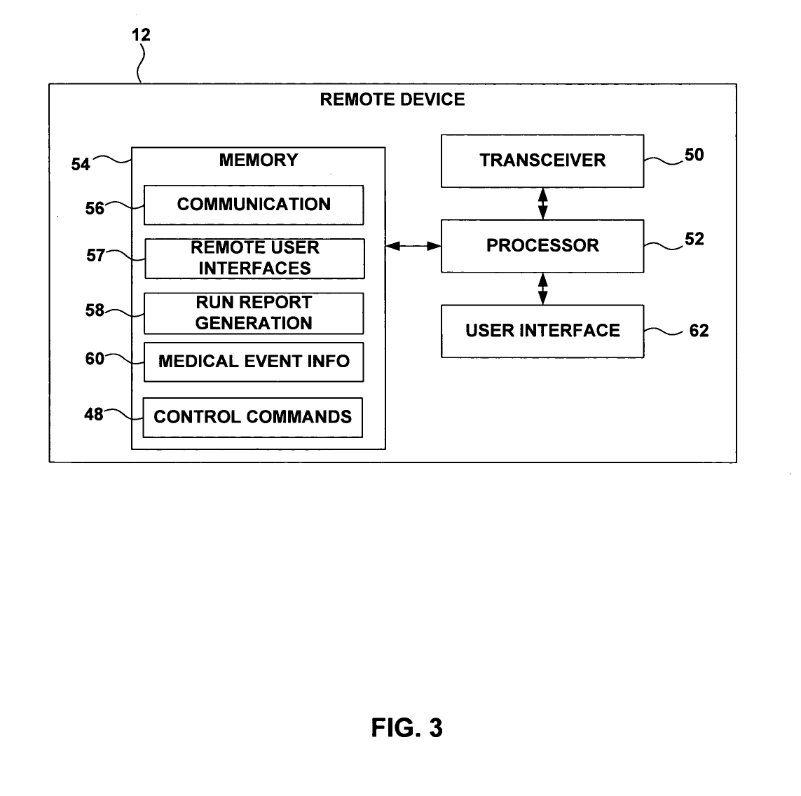

FIG. 3 is a block diagram illustrating an example remote device 12. In the example of FIG. 3, remote device 12 is a computer such as a tablet computer. Remote device 12 wirelessly controls an external medical device, such as defibrillator 14 by sending one or more of control commands 48 to defibrillator 14 via a wireless communication medium.

As illustrated in FIG. 3, remote device 12 includes a transceiver 50 for wireless communication. In one embodiment, transceiver 50 includes an antenna (not shown) to facilitate wireless communication via a radio frequency (RF) communication medium. In this embodiment, transceiver 50 takes, for example, the form of an integrated circuit or PCIMCA card with circuitry configured for wireless communication accordingly, e.g., a wireless network interface card. The wireless network interface card is, in this embodiment, compliant with one or more wireless communication standards, such as to one or both of the Bluetooth or 802.11 specification sets. In another embodiment, transceiver 50 takes the form of IrDA port for communication via the wireless communication medium that includes infrared light waves and compliant with IrDA specification sets. In yet another embodiment, transceiver 50 contains circuitry similar to a cellular telephone to communicate via a RF wireless communication medium.

A processor 52 coupled to transceiver 50 controls operation of transceiver 50. Processor 52 controls transceiver 50 and causes transceiver 20 to detect the presence of an external medical device, establish a wireless communication session, send commands to the external medical device, receive acknowledgments from the external medical device in response to the commands, transmit medical event information and receive medical event information. Processor 52 also generates run reports. Processor 52, in some embodiments, takes the form of a microprocessor.

Processor 52 also accesses a memory 55, which, in some embodiments, includes program instructions that cause processor 52 to perform the functions attributed to processor 52. Memory 54 stores communication program instructions 56, remote user interface program instructions 57, run report generation program instructions 58, medical event info 60, and control commands 48. Communication program instructions 56 cause processor 52 to establish a wireless communication session in with an external medical device. Communication program instructions 56 may further cause processor 52 to detect the presence of a proximate external medical device. Remote user interfaces program instructions 57 cause processor 52 to provide a remote user interface by which an operator can specify one or more control commands 48 to be sent to the external medical device. Run report generation program instructions 58 cause processor 52 to generate a run report, either automatically or in cooperation with an operator, based on medical event information 60. Remote device 12 receives, in some embodiments, at least a portion of medical event information 60 from an external medical device, such as defibrillator 14.

Control commands 48 are commands to control an external medical device, such as defibrillator 14. Processor 52 controls transceiver 50 to send one or more of control commands 48 to defibrillator 14 in response to a selection made by an operator via a user interface 62. User interface 62 may include input-output devices, such as a keyboard, keypad, pointing device, touchscreen, CRT, LED display, LCD display, or speaker. In some embodiments, user interface 62 presents a graphical user interface via a display that resembles the interface of an external medical device to which remote device 12 is connected via a wireless communication session. In this embodiment, processor 52 executes remote user interfaces program instructions 57 that cause processor 52 to control user interface 62 and present the graphical user interface of the external medical device. The graphical user interface, in this embodiment, may include graphical representations of input and output devices of the external medical device, e.g., defibrillator 14. The operator of remote device 12 remotely operates the external medical device by interacting with the graphical representations of the input and output devices.

In response to input from the operator, processor 52 determines an associated command or set of commands 48 to send to the external medical device via the wireless communication session. For example, the operator may use remote device 12 to specify a power level at which defibrillator 14 should apply a defibrillation pulse. Processor 52 receives the input from the operator and generates one or more control commands 48. Processor 52 controls transceiver 50 to send the control commands 48 to defibrillator 14, which carries out the commands and adjusts the power level according to the operator's specification.

In some embodiments, the operator causes processor 52 to send at least a portion of medical event information 60 to an external medical device via an established wireless communication session. The operator can direct also processor 52 to interrogate the external medical device for medical device information via the established wireless communication session. Processor 52 updates medical event information 60 according to medical event information received from the external medical device. In response to any successful transmission via the established wireless communication session, processor 52 may control transceiver 50 to send an acknowledgement to the external medical device indicating the last transmission was successfully received.

After remote device 12 receives the medical event information from the external medical device, the operator may direct the remote device to generate a run report. In response, processor 52 executes run report generation program instructions 58 and generates a run report based on medical event information 60.

Remote device 12 need not be a computer as shown in FIG. 3. Remote device 12 may be, for example, another external medical device, a PDA, a cellular telephone, or a dedicated remote device. Remote device 12 need not include all of the functionality shown in FIG. 3. For example, the invention encompasses embodiments in which remote device 12 lacks the capability to generate run reports.

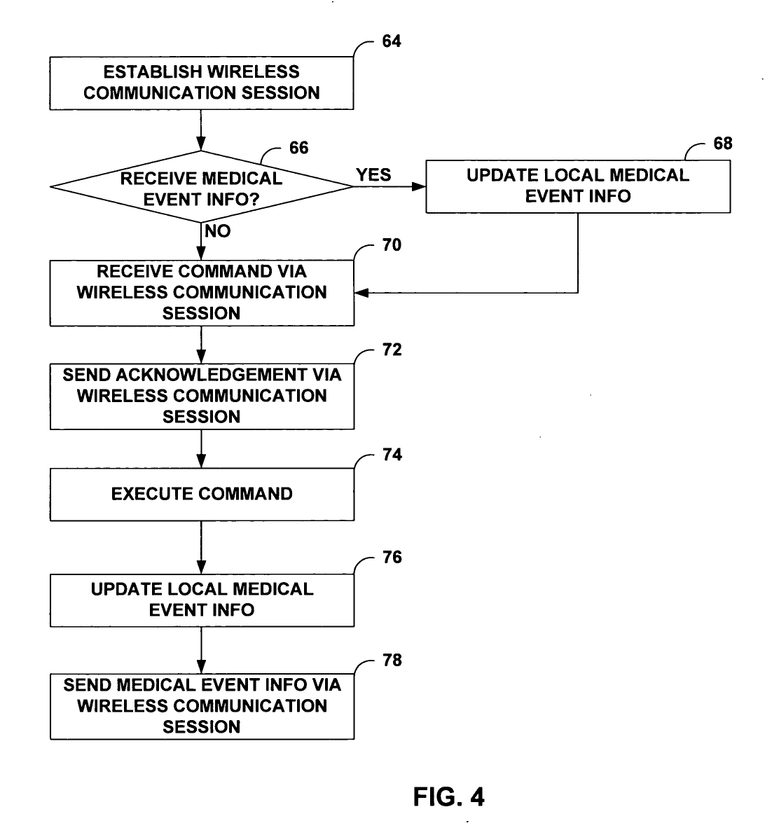

FIG. 4 is a flow diagram illustrating an exemplary process by which an external medical device, such as defibrillator 14, executes remote commands sent by remote device 12 via a wireless communication session. The external medical device establishes a wireless communication session with remote device 12 (64) by any technique. The external medical device may, for example, transmit a paging signal to determine whether a remote device is nearby, or may listen for a paging signal from a remote device, or connect with a network.

Once the wireless communication session is established, the external medical device, in some circumstances, receives medical event information from remote device 12 (66). In the case of defibrillator 14, for example, the age and weight of the patient may be medical event information that is useful to defibrillator 14. In the event the external medical device receives medical event information, the external medical device updates local medical event information 30 with the received medical event information (68).

The external medical device receives a command from remote device 12 via the established wireless communication session (70) and stores the remote command to memory 24. In some embodiments, the external medical device sends an acknowledgement of the received command to remote device 12 via the established wireless communication session to indicate successful receipt of the command (72).

Processor 22 carries out the received command (74). In some embodiments, execution of the command causes processor 22 update local medical event information (76) and to send at least a portion of the medical event information to remote device 12 (78). When the command directs the external medical device to apply therapy, for example, the external medical device may record the therapy applied and the response of the patient to the therapy. Some commands, such as a command to change a display presented by the external medical device, result in no update to local medical event information and do not entail sending medical event information to remote device 12.

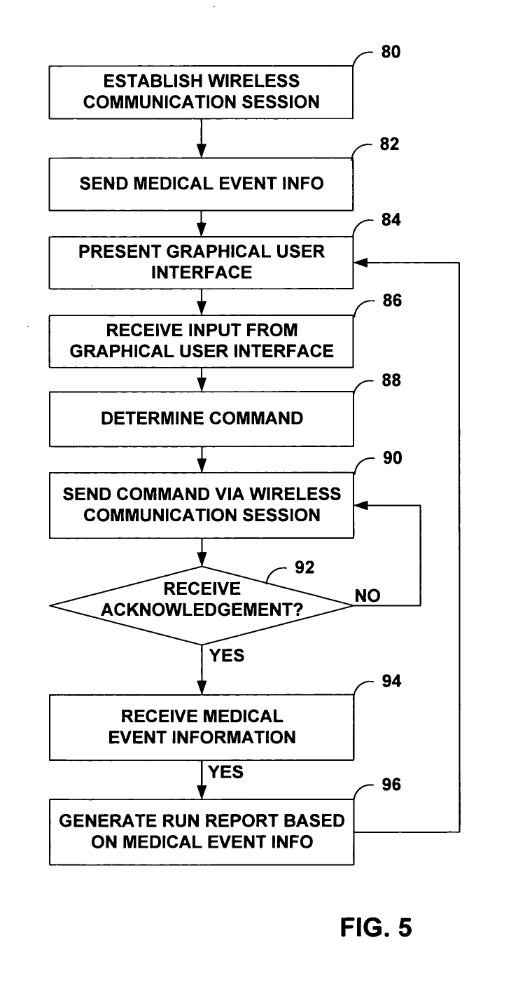

FIG. 5 is a flow diagram illustrating an exemplary process by which remote device 12, such as the computer shown in FIG. 3, remotely controls an external medical device. Remote device 12 establishes a wireless communication session with the external medical device by any technique (80). In some embodiments, remote device 12 sends at least a portion of medical event information 60 to the external medical device via the established wireless communication session (82). In other embodiments, however, remote device 12 does not send medical event information 60 to the external medical device.

Remote device 12 may interact with an operator by presenting the operator with a graphical user interface (84). The operator supplies input to remote device 12 by interacting with the graphical user interface. Remote device 12 receives the input from the operator (86). When the input from operator pertains to controlling the external medical device, remote device processor 52 determine an appropriate command or commands that will carry out the operator's directions (86) and sends the commands to the external medical device (90). In a typical embodiment, remote device 12 waits for an acknowledgement of the command from the external medical device (92). In the event an acknowledgement is not received before a pre-set time expires, remote device 12 resends the command (90).

Remote device 12 may receive medical event information from the external medical device (94). The external medical device may send the medical event information in response to an interrogation from remote device 12, for example, or may send the medical event information after carrying out a command. In some embodiments, remote device 12 generates a run report (96) based on the medical event information received from various sources.

The invention may offer one or more advantages. Various embodiments of the invention offer a great degree of flexibility in operation of an external medical device. Various embodiments also facilitate device-to-device communication without the inconvenience associated with cables or wires. The invention also supports device-to-device communication that enables efficient generation of run reports. In some circumstances, the isolation provided by remote operation of an external medical device may give the operator a safety margin. Moreover, some embodiments support the convenient control of multiple external medical devices with a single remote device.

The preceding specific embodiments are illustrative of the practice of the invention. Various modifications may be made without departing from the scope of the claims. For example, the invention is not limited to remote control of a defibrillator, but may be applied to many other kinds of external medical devices. In addition, the external medical device or the remote device may include more or less functionality than described herein. For example, the invention encompasses embodiments in which the external medical device generates a run report in response to commands from the remote device.

The invention has been described in the context of use by early responders to medical emergencies, such as emergency medical technicians. The invention is not limited to use by operators in the field, however. Embodiments of the invention may be used in a hospital environment, for example.

In addition, the invention may be embodied as a computer-readable medium that includes instructions for causing a programmable processor, such as processors 22 and 52 in FIGS. 2 and 3, to carry out the methods described above. A “computer-readable medium” includes but is not limited to read-only memory, Flash memory and a magnetic or optical storage medium. The instructions may be implemented as one or more software modules, which may be executed by themselves or in combination with other software. These and other embodiments are within the scope of the following claims.

1. A method comprising:
establishing a wireless communication session with an external medical device; and
sending a command to the external medical device via the wireless communication session to remotely operate the external medical device.

2. The method of claim 1, wherein sending a command to remotely operate the external medical device comprises sending a command to change a display presented by the external medical device.

3. The method of claim 1, wherein sending a command to remotely operate the external medical device comprises sending a command to cause the external medical device to apply a therapy.

4. The method of claim 1, wherein sending a command to remotely operate the external medical device comprises sending a command to cause the external medical device to access a memory included in the external medical device.

5. The method of claim 1, wherein sending a command to remotely operate the external medical device comprises sending a command to cause the external medical device to initiate a data transmission to a remote location.

6. The method of claim 1, further comprising receiving an acknowledgement to the command from the external medical device via the wireless communication session.

7. The method of claim 1, further comprising receiving medical event information from the external medical device via the wireless communication session.

8. The method of claim 7, further comprising generating a run report based on the received medical event information.

9. The method of claim 7, wherein the medical event information comprises at least one of an electrocardiogram, a capnogram, a plethysmograph, a heart rate, a blood oxygen saturation, and a blood pressure

10. The method of claim 7, wherein the medical event information comprises at least one of a therapy delivered to the patient, and a time at which the therapy was delivered to the patient.

11. The method of claim 1, further comprising presenting a graphical user interface, wherein an operator interacts with the graphical user interface to specify the command.

12. The method of claim 1, wherein establishing a wireless communication session comprises detecting the external medical device.

13. The method of claim 1, wherein establishing a wireless communication session further comprises establishing the wireless communication session in accordance with one of a Bluetooth specification set, an Infrared Data Association (IrDA) specification set, an 802.11A specification set, an 802.11B specification set and an 802.11G specification set.

14. The method of claim 1, wherein the external medical device comprises at least one of an external defibrillator, an automated external defibrillator (AED), a personal monitor, a drug delivery device, and an automated chest thumper.

15. A method comprising:
establishing a wireless communication session between an external medical device and a remote device;
receiving a command from the remote device via the wireless communication session; and
carrying out the command to operate the external medical device.

16. The method of claim 15, wherein carrying out the command comprises at least one of changing a display, applying a therapy, configuring the therapy, accessing a memory, initiating collection of vital signs, initiating data transmission to a remote location and updating medical event information.

17. The method of claim 15, further comprising sending medical event information to the remote device via the wireless communication session.

18. The method of claim 15, further comprising sending an acknowledgement of the received command to the remote device via the wireless communication medium.

19. The method of claim 15, wherein establishing a wireless communication session comprises detecting the remote device.

20. The method of claim 15, wherein establishing a wireless communication session further comprises establishing the wireless communication session in accordance with one of a Bluetooth specification set, an Infrared Data Association (IrDA) specification set, an 802.11A specification set, an 802.11B specification set and an 802.11G specification set.

21. The method of claim 15, wherein establishing a wireless communication session with a remote device comprises establishing a wireless communication session with one of a computer, a personal digital assistant (PDA), a cellular telephone, and an external medical device.

22. A device comprising:
a transceiver to establish a wireless communication session with an external medical device; and
a processor to control the transceiver to send a command to the medical device via the wireless communication session to remotely operate the external medical device.

23. The device of claim 22, wherein the transceiver is configured to receive medical event information via the wireless communication session, and the processor is configured to generate a run report based on the received medical event information.

24. The device of claim 22, further comprising an input-output device.

25. The device of claim 24, wherein the processor is configured to present a graphical user interface via the input-output device.

26. The device of claim 24, wherein the input-output device comprises at least one of a mouse, a keyboard, a touchscreen, a CRT, an LED display, an LCD display, a microphone and a speaker.

27. The device of claim 22, wherein the transceiver is configured to detect the external medical device.

28. An external medical device comprising:
a transceiver to establish a wireless communication session with a remote device and to receive a command from the remote device; and
a processor to carry out the command to operate the external medical device.

29. The device of claim 28, further comprising a sensor to sense medical data, the sensor comprising at least one of a temperature sensor, a microphone, an ECG monitor, an oxygen sensor, a carbon dioxide sensor, a respiratory sensor, a blood pressure monitor, a twelve-lead electrode set, a five-lead electrode set, and a three-lead electrode set.

30. The device of claim 29, wherein the processor is configured to generate medical event information as a function of the sensed medical data and to control the transceiver to send the medical event information to the remote device via the wireless communication session.

31. The device of claim 28, wherein the device comprises at least one of an electrocardiograph, a capnograph, a plethysmograph, a heart rate monitor, a blood oxygen monitor, a blood pressure monitor, an external defibrillator, an automated external defibrillator (AED), a drug delivery device, and an automated chest thumper.

32. A system comprising:
an external medical device; and
a remote device,
wherein the external medical device is configured to carry out commands sent via a wireless communication session with the remote computing device.

33. The system of claim 32, wherein the external medical device comprises at least one of an electrocardiograph, a capnograph, a plethysmograph, a heart rate monitor, a temperature monitor, a blood oxygen monitor, a blood pressure monitor, an external defibrillator, an automated external defibrillator (AED), a drug delivery device, and an automated chest thumper.

34. The system of claim 32, wherein the remote device comprises at least one of a computer, a personal digital assistant (PDA), a cellular telephone, and a second external medical device.

35. A computer-readable medium comprising instructions for causing a programmable processor to:
control a transceiver to establish a wireless communication session with an external medical device; and
send a command to the external medical device via the wireless communication session to remotely operate the external medical device.

36. The medium of claim 35, wherein the instructions further cause the processor to generating a run report based on medical event information received from the external medical device via the wireless communication session.

37. A computer-readable medium comprising instructions for causing a programmable processor to:
control a transceiver to establish a wireless communication session with a remote device; and
carry out a command received from the remote device via the wireless communication session.

38. The medium of claim 37, wherein the instructions causing the processor to carry out a command further cause the processor to change a display.

39. The medium of claim 37, wherein the instructions causing the processor to carry out a command further cause the processor to apply a therapy.

40. The medium of claim 37, wherein the instructions causing the processor to carry out a command further cause the processor to initiate a data transmission to a remote location.

41. The medium of claim 37, wherein the instructions further cause the processor to generate medical event information as a function of medical data sensed via a sensor, and to control the transceiver to send the medical event information to the remote device via the wireless communication session.
